(19) United States
(12) Patent Application Publication
(10) Pub. No.: US 2013/0137913 A1
(43) Pub. Date: May 30, 2013

(54) PROCESS FOR THE REJUVENATION OF A SPENT MOLECULAR SIEVE CATALYST

(71) Applicant: SHELL OIL COMPANY, Houston, TX (US)

(72) Inventors: Leslie Andrew CHEWTER, (US); Marianne Hendrina Francisca KOX, (US); Jeroen VAN WESTRENEN, (US); Ferry WINTER, (US)

(73) Assignee: SHELL OIL COMPANY, Houston, TX (US)

(21) Appl. No.: 13/687,556

(22) Filed: Nov. 28, 2012

(30) Foreign Application Priority Data
Nov. 28, 2011 (EP) 11190983.4

(51) Int. Cl. B01J 29/90; C07C 4/06; C07C 1/20

(52) U.S. Cl. CPC B01J 29/90; C07C 1/20; C07C 4/06
USPC 585/638; 585/651; 585/653; 502/27; 502/28

(57) ABSTRACT

The present invention relates to a process for the rejuvenation of a spent molecular sieve, comprising at least the steps of:
(a) treating the spent molecular sieve catalyst with an aqueous solution comprising at least one acid to provide a rejuvenated molecular sieve catalyst and a spent aqueous solution;
(b) removing spent aqueous solution from the rejuvenated molecular sieve catalyst to provide the rejuvenated molecular sieve catalyst.

This application claims the benefit of European Patent Application No. 11190983.4, filed on Nov. 28, 2011, the disclosure of which is incorporated by reference herein in its entirety.

### FIELD OF THE INVENTION

The present invention relates to a process for the rejuvenation of a spent molecular sieve catalyst, such as a molecular sieve catalyst which has been used as a catalyst in the conversion of one or both of oxygenates to olefins and an olefin cracking process, and the use of such a process in processes for the preparation of an olefinic product, such as one or both of ethylene and propylene.

### BACKGROUND OF THE INVENTION

Conventionally, ethylene and propylene are produced via steam cracking of paraffinic feedstocks including ethane, propane, naphtha and hydrowax. An alternative route to ethylene and propylene is an oxygenate-to-olefin (OTO) process. Interest in OTO processes for producing ethylene and propylene is growing in view of the increasing availability of natural gas. Methane in the natural gas can be converted into for instance methanol or dimethylether (DME), both of which are suitable feedstocks for an OTO process.

In an OTO process, an oxygenate such as methanol is provided to a reaction zone of a reactor comprising a suitable conversion catalyst and converted to ethylene and propylene. In addition to the desired ethylene and propylene, a substantial part of the methanol is converted to higher hydrocarbons including C4+ olefins and paraffins. In order to increase the ethylene and propylene yield of the process, the C4+ olefins may be recycled to the reaction zone or alternatively further cracked in a dedicated olefin cracking zone to produce further ethylene and propylene.

Conversion catalysts utilised in OTO processes can comprise molecular sieve. The catalytic activity of such molecular sieve catalysts can decrease over time, in part due to the build up of carbonaceous deposits on the molecular sieve catalyst, and in particular on the molecular sieve in the catalyst, due to side reactions arising from the decomposition of the oxygenate feedstock at the OTO process temperatures. As a consequence the molecular sieve catalyst becomes deactivated.

Such deactivated molecular sieve catalyst comprising carbonaceous deposits may be periodically regenerated by heating the deactivated molecular sieve catalyst with an oxidant-comprising gas in order to remove at least a portion of the carbonaceous deposits as gaseous oxides of carbon, such as carbon monoxide and carbon dioxide. Such a regeneration process can restore a portion of the lost catalytic activity. However over time the effectiveness of the regeneration decreases, until the catalytic activity of the regenerated molecular sieve catalyst becomes so low that it is no longer viable and the so-called spent molecular sieve catalyst is replaced with fresh molecular sieve catalyst.

U.S. Patent Publication No. 2008/0015402 discloses a method of rejuvenating a molecular sieve catalyst that has decreased catalytic activity as a result of contact with moisture. The catalyst is used to make an olefin product from methanol feed. The molecular sieve catalyst can be rejuvenated by heating at a rate sufficient to increase the catalytic activity of the molecular sieve catalyst. The molecular sieve catalyst is heated at a heat rate of greater than 40° C./min until it has a cumulative grams of methanol converted per gram of sieve that is increased by at least 5% relative to that at a heat rate basis of 40° C./min over a temperature range of from 25° C. to 475° C.

The rejuvenation process of U.S. Patent Publication No. 2008/0015402 requires the heating of the molecular sieve catalyst and associated OPEX costs.

A need therefore exists to provide an alternative process for the rejuvenation of a spent molecular sieve catalyst which does not require the heating of the spent molecular sieve catalyst to 475° C. Such a regeneration process should increase the catalytic activity of the rejuvenated molecular sieve catalyst compared to the spent molecular sieve catalyst. Preferably, the regeneration process should restore, at least in part, and preferably completely, the catalytic activity of a spent molecular sieve catalyst to that of a fresh molecular sieve catalyst without compromising the selectivity.

### SUMMARY OF THE INVENTION

It has surprisingly been found that a spent molecular sieve catalyst, such as molecular sieve catalyst that was previously used in an oxygenate-to-olefin or an olefin cracking process, can be rejuvenated by treatment with an aqueous solution comprising at least one acid. The rejuvenation treatment leads to a significant increase in the catalytic activity of the molecular sieve catalyst, compared to that of the spent molecular sieve catalyst.

Furthermore, the rejuvenation treatment does not increase the catalytic activity at the expense of selectivity.

In a first aspect, the present invention provides a process for rejuvenating a spent molecular sieve catalyst, said process comprising at least the steps of:

treating the spent molecular sieve catalyst with an aqueous solution comprising at least one acid to provide a rejuvenated molecular sieve catalyst and a spent aqueous solution;

removing spent aqueous solution from the rejuvenated molecular sieve catalyst to provide the rejuvenated molecular sieve catalyst.

Reference herein to a molecular sieve catalyst is to a catalyst comprising molecular sieve. Reference herein to spent molecular sieve catalyst is to catalyst which is subject to a loss of catalytic activity, which loss of activity is not restored by an oxidative regeneration. In one embodiment, the molecular sieve comprised in the catalyst may be selected from the group comprising silicoaluminophosphate and aluminosilicate. The molecular sieve is preferably an aluminosilicate, such as a zeolite. In another embodiment the molecular sieve is zeolite having in at least one direction a 10-membered ring structure. In yet another embodiment, the zeolite may comprise one or more of the group comprising a TON-type aluminosilicate, such as ZSM-22, a MTT-type aluminosilicate, such as ZSM-23, MFI-type aluminosilicate, such as ZSM-5, and MEL-type aluminosilicate, such as ZSM-11.

In a further embodiment, the spent molecular sieve catalyst may be one which has been used as catalyst in the catalytic conversion of oxygenates into olefins (OTO) and/or an olefin cracking process (OCP). As defined herein, the term ‘used as a catalyst’ means that the molecular sieve has been exposed to a feedstock, such as an oxygenate feedstock or an olefin feedstock and used to catalyse the conversion of the feedstock to one or more products, such as ethylene and propylene

For instance, the spent molecular sieve catalyst may have been obtained from one or both of an oxygenate to olefin process and an olefin cracking process, and can be provided by one or both of the steps of:
(i) reacting a feedstock comprising oxygenate in an oxygenate to olefin reaction zone in the presence of a molecular sieve catalyst to produce a spent molecular sieve catalyst and a reaction effluent comprising olefins; and
(ii) reacting a C4+ hydrocarbon feedstock comprising olefin in an olefin cracking process reaction zone in the presence of a molecular sieve catalyst to produce a spent molecular sieve catalyst and a reaction effluent comprising one or both of ethylene and propylene.
   As used herein, the term “Cn+” means an organic compound comprising n or more carbon atoms. For instance, a C4+ hydrocarbon comprises 4 or more carbon atoms.

The spent molecular sieve catalyst may have a catalytic activity, for instance a catalytic activity for one or both of the conversion of oxygenate to olefin and the conversion of C4+ olefin to one or both of ethylene and propylene in an olefin cracking process, which is less than that of fresh catalyst of identical composition. Typically the catalytic activity is measured on the basis of one or more specific olefin products, more typically the C5 olefins. In another embodiment, the spent catalyst comprising spent molecular sieve catalyst may have a catalytic activity of less than 80% of that of fresh molecular sieve catalyst, more typically less than 50%. The rejuvenated molecular sieve catalyst may have a catalytic activity of greater than 50% of that of the fresh molecular sieve catalyst, more typically greater than 70%, still more typically greater than 90%.

As used herein, the term ‘fresh molecular sieve catalyst’ means molecular sieve catalyst in the form immediately before it is exposed to a feedstock for the first time. As such, the catalytic activity of fresh molecular sieve catalyst should be measured after any processing steps which could affect activity, such as any ion exchange steps or steps which would increase the acidity of the molecular sieve catalyst, which may be applied prior to the molecular sieve catalyst being contacted with feedstock for the first time. For the avoidance of doubt, it is pointed out that the spent molecular sieve catalyst is the product of the fresh molecular sieve catalyst after use in the catalytic conversion of a feedstock.

In yet another embodiment, the at least one acid in the aqueous solution may comprise at least one carboxylic acid group, and preferably at least two carboxylic acid groups. The at least one acid may be a carboxylic acid, such as one or more selected from the group comprising acetic acid, oxalic acid and tartaric acid, with oxalic acid being preferred.

In another embodiment, the at least one acid may comprise an acidified ammonia solution. The acidified ammonia solution may be prepared from the acidification of an aqueous ammonia solution with a strong acid. As used herein, the term ‘strong acid’ is defined as an acid which can undergo substantially complete ionisation of its most acidic proton in aqueous solution at pH 2 and lower, for instance an acid with a pKₐ below 2.0. Preferably, the strong acid is one or more of the group comprising hydrochloric acid, hydrobromic acid, hydroiodic acid, nitric acid, sulphuric acid and p-toluene sulphonic acid, more preferably hydrochloric acid. In a preferred embodiment, the ammonium salt of a strong acid may be selected from the group comprising ammonium nitrate, ammonium chloride and ammonium sulphate.

In a still further embodiment, the step of treating the spent molecular sieve catalyst may comprise contacting the spent molecular sieve catalyst with an aqueous solution comprising at least one acid. The acid may be present in the aqueous solution at a concentration in the range of from 0.01 to 3 M, preferably 0.05 to 2 M and more preferably 0.075 to 1.25 M.

In yet another embodiment, the step of removing spent aqueous solution from the rejuvenated molecular sieve catalyst may comprise heating the rejuvenated molecular sieve catalyst. The rejuvenated molecular sieve catalyst may be heated to a temperature sufficient to drive off the spent aqueous solution, for instance a temperature above 100° C., more preferably a temperature above 200° C. The temperature should be less than 700° C., more preferably less than 550° C., in order to avoid damage to the framework of the molecular sieve.

In another embodiment, the rejuvenated molecular sieve catalyst may be washed with a wash solution comprising water before the step of heating the rejuvenated molecular sieve catalyst. The water in the wash solution is preferably de-ionised in order to minimise ion exchange with molecular sieve in the catalyst. The washing step can remove at least a portion, preferably substantially all, of the spent aqueous solution. This step can reduce deposition of solid residue from the spent aqueous solution on the rejuvenated molecular sieve catalyst during the heating step.

In yet another embodiment, the molecular sieve catalyst may be regenerated to remove at least a part of any carbonaceous material deposited as a result of the oxygenate conversion, before being provided to step (a) of the process according o the first instance. The process according to the first instance may thus preferably further comprise, before the step of treating the spent molecular sieve catalyst with an aqueous solution, the step of:

regenerating the spent molecular sieve catalyst to remove at least a part of any carbonaceous deposits on the spent molecular sieve catalyst by heating with a regeneration gas stream comprising oxidant.

Alternatively, the process according to the first instance may preferably further comprise, after the step of removing spent aqueous solution from the rejuvenated molecular sieve catalyst, the step of:

regenerating the rejuvenated molecular sieve catalyst to remove at least a part of any carbonaceous deposits by heating with a regeneration gas stream comprising oxidant.

The regeneration step removes deposited carbonaceous material from one or both of the surface and the pores of the molecular sieve. The regeneration step may comprise heating the spent molecular sieve catalyst in a regenerating medium, such as a gas comprising oxygen, to a temperature sufficient to burn off carbonaceous material. Typically, the rejuvenated molecular sieve catalyst can be heated to a temperature in the range of from 250 to 700° C., more typically in the range of from 300 to 650° C.

In a further embodiment, the step of removing the spent aqueous solution from the rejuvenated molecular sieve catalyst may be combined with the conversion process in which it is to be used, such as one or both of an oxygenate to olefin process and an olefin cracking process. Both processes may operate at temperatures in a range suitable to remove the spent aqueous solution. Typically, the rejuvenated molecular sieve catalyst can be heated to a temperature in the range of from 250 to 600° C. Thus, the step of removing the spent aqueous solution from the rejuvenated molecular sieve catalyst may be carried out in the reaction zone. The small amount of acids which may still be present in the catalyst will not negatively influence the performance of the catalyst nor will is negatively influence the reaction.

In a still further embodiment, the step of removing the spent aqueous solution from the rejuvenated molecular sieve catalyst may be combined with regeneration to remove carbonaceous material. For instance, the rejuvenated molecular sieve catalyst may be heated with a regeneration gas comprising an oxidant, such as oxygen, to a temperature sufficient to burn off carbonaceous material and drive off the spent aqueous solution and/or solution comprising water. Thus, the step of removing the spent aqueous solution from the rejuvenated molecular sieve catalyst may be carried out in the regeneration zone. Typically, regeneration temperatures may be greater than 500° C., which is significantly higher than that required to remove the spent aqueous solution. Consequently, this embodiment is less preferred than that of the previous paragraph.

In a second aspect, the present invention provides a process for the preparation of an olefinic product, the process comprising at least the steps of:
(a) reacting a feedstock comprising oxygenate in an oxygenate to olefin reaction zone in the presence of a molecular sieve catalyst to produce a reaction effluent comprising, olefins and a spent molecular sieve catalyst;
(b) rejuvenating the spent molecular sieve catalyst in accordance with a process according to the first aspect and its embodiments discussed above to provide a rejuvenated molecular sieve catalyst.

In a further embodiment, after rejuvenating the spent molecular sieve catalyst, the process may further comprise the steps of:
returning the rejuvenated catalyst comprising rejuvenated molecular sieve catalyst to the reaction zone.

In a third aspect, the present invention provides a process for the preparation of olefinic product comprising one or both of ethylene and propylene, the process comprising at least the steps of:
(a) reacting a C4+ hydrocarbon feedstock comprising olefin in an olefin cracking process reaction zone in the presence of a molecular sieve catalyst to produce a spent molecular sieve catalyst and a reaction effluent comprising one or both of ethylene and propylene;
(b) rejuvenating the spent molecular sieve catalyst according to a process of the first aspect and its embodiments discussed above to provide a rejuvenated molecular sieve catalyst.

In a fourth aspect, the present invention provides, a process for the preparation of olefinic product, the process comprising at least the steps of:
(a) (1) reacting an C4+ hydrocarbon feedstock comprising olefin in an olefin cracking process reaction zone in the presence of a molecular sieve catalyst to produce a reaction effluent stream comprising one or both of ethylene and propylene;
(2) passing at least part of the catalyst used in step (1) to an oxygenate to olefin reaction zone;
(3) reacting a feedstock comprising oxygenate in the oxygenate to olefin reaction zone in the presence of the molecular sieve catalyst of step (1) to produce a spent molecular sieve catalyst and a reaction effluent stream comprising olefins;
(b) rejuvenating the spent molecular sieve catalyst according to a process of the first aspect and its embodiments discussed above to provide a rejuvenated molecular sieve catalyst.

In one embodiment of the second, third or fourth aspects, the molecular sieve is aluminosilicate molecular sieve, and more particularly zeolite molecular sieve.

In another embodiment of the second, third or fourth aspects, after the step of treating the spent molecular sieve catalyst with an aqueous solution, typically after removing spent aqueous solution from the rejuvenated molecular sieve catalyst, the process may further comprise the steps of:
returning the rejuvenated catalyst comprising rejuvenated molecular sieve catalyst to the reaction zone.

In a further embodiment of the second, third or fourth aspects, the rejuvenation process may be carried out as a batch, semi-continuous or continuous process. In a continuous process, spent catalyst can be removed from the reaction or regeneration zones as a fluidised spent catalyst stream and passed to a rejuvenation zone for treatment. The aqueous solution and spent aqueous solutions can be continuously added and removed respectively from the rejuvenation zone. Rejuvenated catalyst can then be returned to the reaction zone or regeneration zone as a fluidised rejuvenated catalyst stream. The rejuvenated catalyst returned to the reaction or regeneration zones may still comprise spent aqueous solution, such that the removal of the spent aqueous solution, or wash solution comprising water may be carried out in the reaction or regeneration zones.

### DETAILED DESCRIPTION OF THE INVENTION

Embodiments of the present invention will now be described by way of example only.

The spent molecular sieve catalyst which is rejuvenated in the process described herein can be produced during the catalytic conversion of an oxygenate feedstock to olefinic products in an oxygenate-to-olefins process or during the catalytic cracking of a C4+ hydrocarbon feedstock comprising olefin in an olefin cracking process.

For instance, an oxygenate feedstock stream can be contacted in an OTO reaction zone, such as an OTO reactor, with a catalyst for oxygenate conversion under oxygenate conversion conditions, to obtain a reaction effluent comprising olefins, particularly lower olefins. Reference herein to an oxygenate feedstock is to an oxygenate—comprising feedstock. In the OTO reaction zone, at least part of the feedstock is converted into a product containing one or more olefins, preferably including lower olefins, in particular ethylene and typically propylene.

The reaction effluent can be removed from OTO reaction zone as reaction effluent stream. Reaction effluent stream may comprise unreacted oxygenate, olefinic product and water and can be processed in a variety of ways known in the art.

The oxygenate used in the process is preferably an oxygenate which comprises at least one oxygen-bonded alkyl group. The alkyl group preferably is a C1-C5 alkyl group, more preferably C1-C4 alkyl group, i.e. comprises 1 to 5, or 1 to 4 carbon atoms respectively; more preferably the alkyl group comprises 1 or 2 carbon atoms and most preferably one carbon atom. Examples of oxygenates that can be used in the oxygenate feedstock include alcohols and ethers. Examples of preferred oxygenates include alcohols, such as methanol, ethanol, propanol; and dialkyl ethers, such as dimethylether, diethylether, methylethylether. Preferably, the oxygenate is methanol or dimethylether, or a mixture thereof.

Preferably the oxygenate feedstock comprises at least 50 wt % of oxygenate, in particular methanol and/or dimethylether, based on total hydrocarbons, more preferably at least 70 wt %.

The oxygenate feedstock can comprise an amount of diluent, such as water or steam. In one embodiment, the molar ratio of oxygenate to diluent is between 10:1 and 1:10, preferably between 4:1 and 1:2, in particular when the oxygenate is methanol and the diluent is water (typically steam).

Preferably, in addition to the oxygenate, an olefinic co-feed is provided along with and/or as part of the oxygenate feedstock. The co-feed may be supplied to OTO reaction zone as an olefinic co-feed stream, or injected into the oxygenate feedstock stream discussed above. Reference herein to an olefinic co-feed is to an olefin-comprising co-feed. The olefinic co-feed preferably comprises C4 and higher olefins, more preferably C4 and C5 olefins. Preferably, the olefinic co-feed comprises at least 25 wt %, more preferably at least 50 wt %, of C4 olefins, and at least a total of 70 wt % of C4 hydrocarbon species.

Preferably, at least 70 wt % of the olefinic co-feed, during normal operation, is formed by a recycle stream of a C4+hydrocarbon fraction from the OTO reaction effluent. Preferably at least 90 wt % of olefinic co-feed, based on the whole olefinic co-feed, is formed by such recycle stream. In order to maximize production of ethylene and propylene, it is desirable to maximize the recycle of C4 olefins in the effluent of the OTO process. This can be done by recycling at least part of the C4+ hydrocarbon fraction, preferably C4-C5 hydrocarbon fraction, more preferably C4 hydrocarbon fraction, in the OTO effluent. However, a certain part thereof, such as between 1 and 5 wt %, can be withdrawn as purge, since otherwise saturated hydrocarbons, in particular C4s (butane) would build up in the process, which are substantially not converted under the OTO reaction conditions.

The preferred molar ratio of oxygenate in the oxygenate feedstock to olefin in the olefinic co-feed provided to the OTO conversion zone depends on the specific oxygenate used and the number of reactive oxygen-bonded alkyl groups therein. Preferably the molar ratio of oxygenate to olefin in the total feed lies in the range of 20:1 to 1:10, more preferably in the range of 18:1 to 1:5, still more preferably in the range of 15:1 to 1:3, even still more preferably in the range of 12:1 to 1:3.

A variety of OTO processes are known for converting oxygenates, such as for instance methanol or dimethylether to an olefin-containing product, as already referred to above. One such process is described in WO-A 2006/020083. Processes integrating the production of oxygenates from synthesis gas and their conversion to light olefins are described in US20070203380A1 and US20070155999A1.

Catalysts suitable for converting the oxygenate feedstock comprise molecular sieve. Such molecular sieve catalysts, i.e. molecular sieve-comprising catalysts, typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing tetrahedral units, more preferably, two or more [SiO₄], [AlO₄] and/or [PO₄] tetrahedral units. These silicon, aluminum and/or phosphorus based molecular sieves and metal containing silicon, aluminum and/or phosphorus based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å.

Suitable molecular sieves are silicoaluminophosphates (SAPO), such as SAPO-17, -18, -34, -35, -44, but also SAPO-5, -8, -11, -20, -31, -36, -37, -40, -41, -42, -47 and -56; aluminophosphates (A1PO) and metal substituted (silico)aluminophosphates (MeAlPO), wherein the Me in MeAlPO refers to a substituted metal atom, including metal selected from one of Group IA, IIA, IB, IIIB, IVB, VB, VIB, VIIB, VIIIB and Lanthanides of the Periodic Table of Elements. Preferably Me is selected from one of the group consisting of Co, Cr, Cu, Fe, Ga, Ge, Mg, Mn, Ni, Sn, Ti, Zn and Zr.

Alternatively, the conversion of the oxygenate feedstock may be accomplished by the use of an aluminosilicate-comprising catalyst, in particular a zeolite-comprising catalyst. Suitable catalysts include those containing a zeolite of the ZSM group, in particular of the MFI type, such as ZSM-5, the MTT type, such as ZSM-23, the TON type, such as ZSM-22, the MEL type, such as ZSM-11, and the FER type. Other suitable zeolites are for example zeolites of the STF-type, such as SSZ-35, the SFF type, such as SSZ-44 and the EU-2 type, such as ZSM-48.

Aluminosilicate-comprising catalyst, and in particular zeolite-comprising catalyst are preferred when an olefinic co-feed is fed to the oxygenate conversion zone together with oxygenate, for increased production of ethylene and propylene.

Preferred catalysts comprise a more-dimensional zeolite, in particular of the MFI type, more in particular ZSM-5, or of the MEL type, such as zeolite ZSM-11. Such zeolites are particularly suitable for converting olefins to ethylene and/or propylene. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably, the channels in at least one of the directions are 10-membered ring channels. A preferred MFI-type zeolite has a silica-to-alumina ratio, SAR, of at least 60, preferably at least 80. Catalysts may include catalysts comprising one or more zeolites having one-dimensional 10-membered ring channels, i.e. one-dimensional 10-membered ring channels, which are not intersected by other channels. Preferred examples are zeolites of the MTT and/or TON type. In a particularly preferred embodiment the catalyst comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The catalyst may further comprise phosphorus as such or in a compound, i.e. phosphorus other than any phosphorus included in the framework of the molecular sieve. It is preferred that a MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05 to 10 wt % based on the weight of the formulated catalyst. A particularly preferred catalyst comprises phosphorus-treated MEL or MFI-type zeolite having SAR of in the range of from 60 to 150, more preferably of from 80 to 100. An even more particularly preferred catalyst comprises phosphorus-treated ZSM-5 having SAR of in the range of from 60 to 150, more preferably of from 80 to 100.

It is preferred that the molecular sieves in the hydrogen form are used in the oxygenate conversion catalyst, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve used is in the hydrogen form. It is well known in the art how to produce such molecular sieves in the hydrogen form.

The reaction conditions of the oxygenate conversion include a reaction temperature of 350 to 1000° C., preferably from 350 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar).

Preferably, the oxygenate feedstock is preheated to a temperature in the range of from 200 to 550° C., more preferably 250 to 500° C. prior to contacting with the molecular sieve-comprising catalyst.

The catalyst particles used in the process can have any shape known to the skilled person to be suitable for this purpose, and can be present in the form of spray dried catalyst particles, spheres, tablets, rings, extrudates, etc. Extruded catalysts can be applied in various shapes, such as, cylinders and trilobes. Spray-dried particles allowing use in a fluidized bed or riser reactor system are preferred. Spherical particles are normally obtained by spray drying. Preferably the average particle size is in the range of 1-200 μm, preferably 50-100 μm, still more preferably approximately 60-80 μm.

Although the C4+ hydrocarbon fraction in the reaction effluent may be recycled as an olefinic co-feed, at least part of the olefins in the C4+ hydrocarbon fraction may be converted to ethylene and/or propylene by contacting the C4+ hydrocarbon fraction in a separate unit with a molecular sieve-comprising catalyst, particularly a zeolite-comprising catalyst. This is particularly preferred where molecular sieve-comprising catalyst in the OTO process comprises a least one SAPO, AlPO, or MeAlPO type molecular sieve, preferably SAPO-34. These catalysts are less suitable for converting olefins. Preferably, the C4+ hydrocarbon fraction is contacted with the zeolite-comprising catalyst at a reaction temperature of 350 to 1000° C., preferably from 375 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar). Optionally, the stream comprising C4+ olefins also contains a diluent. Examples of suitable diluents include, but are not limited to, liquid water or steam, nitrogen, argon and methane. Under these conditions, at least part of the olefins in the C4+ hydrocarbon fraction are converted to further ethylene and/or propylene. The further ethylene and/or propylene may be combined with the further ethylene and/or propylene obtained directly from the OTO reaction zone. Such a separate process step directed at converting C4+ olefins to ethylene and propylene is also referred to as an olefin cracking process (OCP).

Catalysts comprising molecular sieve, particularly aluminosilicate-comprising catalysts, and more particularly zeolite-comprising catalysts, have the further advantage that in addition to the conversion of methanol or ethanol, these catalysts also induce the conversion of olefins to ethylene and/or propylene. Therefore, aluminosilicate-comprising catalysts, and in particular zeolite-comprising catalysts, are particularly suitable for use as the catalyst in an OCP. The preferences provided herein above for the oxygenate to olefins catalyst apply mutatis mutandis for the OCP catalyst with the primary exception that the OCP catalyst always comprises at least one zeolite.

Particular preferred catalysts for the OCP reaction, i.e. converting part of the olefinic product, and preferably part of the C4+ hydrocarbon fraction of the olefinic product including olefins, are catalysts comprising at least one zeolite selected from MFI, MEL, TON and MTT type zeolites, more preferably at least one of ZSM-5, ZSM-11, ZSM-22 and ZSM-23 zeolites.

The catalyst may further comprise phosphorus as such or in a compound, i.e. phosphorus other than any phosphorus included in the framework of the molecular sieve. It is preferred that a MEL or MFI-type zeolite comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05 to 10 wt % based on the weight of the formulated catalyst. A particularly preferred catalyst comprises phosphorus-treated MEL or MFI-type zeolite having SAR of in the range of from 60 to 150, more preferably of from 80 to 100. An even more particularly preferred catalyst comprises phosphorus-treated ZSM-5 having SAR of in the range of from 60 to 150, more preferably of from 80 to 100.

Preferably, the oxygenate to olefins catalyst and the olefin cracking catalyst are the same zeolite comprising catalyst.

Both the OTO process and the OCP may be operated in a fluidized bed or moving bed, e.g. a fast fluidized bed or a riser reactor system, and also in a fixed bed reactor or a tubular reactor. A fluidized bed or moving bed, e.g. a fast fluidized bed or a riser reactor system are preferred.

In one embodiment, the catalyst comprising molecular sieve may be first used in an OCP reaction zone for the conversion of the C4+ olefins of the C4+ hydrocarbon fraction, and subsequently transferred to the OTO reaction zone for conversion of the oxygenate feedstock stream and olefinic co-feed stream.

The catalyst can deactivate in the course of the OTO process and OCP due in part to the deposition of carbonaceous deposits, such as coke, on the catalyst by side reactions, to produce molecular sieve catalyst comprising carbonaceous deposits, such as coke. The carbonaceous deposits lead to deactivation because they can block the access of the feedstock to the active sites of the molecular sieve. In the process disclosed herein, carbonaceous deposits are formed on the catalyst during an OTO or OCP process, irrespective of whether the catalyst is fresh, spent and/or rejuvenated molecular sieve catalyst.

The molecular sieve catalyst comprising carbonaceous deposits can be oxidatively regenerated to remove a portion of the carbonaceous deposits. It is not necessary, and indeed may be undesirable, to remove all the carbonaceous deposit from the molecular sieve catalyst as it is believed that a small amount of residual carbonaceous deposit such as coke may enhance the catalyst performance. Additionally, it is believed that complete removal of the carbonaceous deposit may also lead to degradation of the molecular sieve in the catalyst.

The same molecular sieve catalyst molecular sieve may be used for both the OTO process and OCP. In such a situation, the molecular sieve catalyst, particularly comprising aluminosilicate molecular sieve and more particularly comprising zeolite, may be first used in the OCP. The catalyst from the OCP may then be used, typically without oxidative regeneration, in the OTO process. The molecular sieve catalyst from the OTO process may comprise carbonaceous deposits and require regeneration as described herein, with the regenerated molecular sieve catalyst then used again in the OCP.

This line-up may be beneficial because it provides good heat integration between the OCP, OTO and oxidative regeneration processes. The OCP is endothermic and at least a portion of the heat of reaction can be provided by passing catalyst from the regeneration zone to the OCP reaction zone, because the regeneration reaction which oxidizes the carbonaceous deposits from the deactivated molecular sieve catalyst is exothermic.

The oxidative regeneration process can be carried out in a regeneration zone. The molecular sieve catalyst comprising carbonaceous deposits may be transferred from the reaction zone to the regeneration zone as a fluidised stream.

The regeneration zone may be a regenerator such as a fluidized bed or moving bed, e.g. a fast fluidized bed or a riser reactor system. Oxidative regeneration may also be carried out in the reaction zone, particularly the reactor itself, such as in a fixed bed reactor or a tubular reactor, although this is not preferred.

The oxidative regeneration can heat the molecular sieve catalyst comprising carbonaceous deposits in an oxidising environment supplied by a regeneration gas stream comprising oxidant. The oxidant may be one or more of the group selected from oxygen, ozone, sulphur trioxide, and NO (such as N₂O, NO, NO₂ and/or N₂O₅). Oxygen is preferred, such that the regeneration gas stream may comprise air. The air may be diluted with a diluent such as nitrogen or carbon dioxide, if required.

The regeneration step can oxidise the carbonaceous deposits on the molecular sieve catalyst to produce gaseous oxides of carbon, which can be removed from the regeneration zone as a regeneration effluent stream which may further comprise any unreacted components of the treated regeneration gas stream, such as any unreacted oxidant, and any inert components, such as nitrogen and/or carbon dioxide already present in the regeneration gas stream e.g. if air is used.

The oxidative regeneration step can be carried out at a regeneration temperature in the range of from 550 to 750° C., more preferably in the range of from 600 to 650° C.

However, it has been found that over time, the regeneration of the molecular sieve catalyst, i.e. removing any carbonaceous deposits from the catalyst, does not return the catalytic activity of a molecular sieve catalyst to that of a fresh catalyst. The rejuvenation process described herein seeks to overcome these problems, by increasing the catalytic activity without compromising selectivity towards that exhibited by a fresh catalyst comprising fresh molecular sieve catalyst.

The rejuvenation process can be carried out on a spent molecular sieve catalyst. This may be a spent molecular sieve catalyst which has been regenerated or a spent molecular sieve catalyst which has not or only partially been regenerated i.e. spent molecular sieve catalyst comprising carbonaceous deposits. Preferably, the rejuvenation process is carried out on regenerated molecular sieve catalyst.

The rejuvenation process can be carried out in a rejuvenation zone, which may be a zone separate from the reaction zone or the regeneration zone. The rejuvenation zone is preferably a reactor wherein the catalyst is suspended in a liquid phase, preferably an aqueous liquid phase. The rejuvenation zone can be supplied with spent molecular sieve catalyst for instance by a fluidized spent molecular sieve catalyst stream from, for example the reaction zone or the regeneration zone. It will be apparent that spent molecular sieve catalyst supplied from the reaction zone without prior regeneration or which has only been partially regenerated may comprise carbonaceous deposits.

The spent molecular sieve catalyst in the rejuvenation zone is treated with an aqueous solution comprising at least one acid, which may for instance be passed to the rejuvenation zone an aqueous solution stream. The step of treating the spent molecular sieve catalyst may comprise contacting the spent molecular sieve catalyst with an aqueous solution stream comprising at least one acid.

The water forming the aqueous solution is preferably de-ionised water, in order to minimise the possibility of the molecular sieve in the catalyst becoming contaminated by solvated species.

The at least one acid may comprise at least one carboxylic acid group, and preferably at least two carboxylic acid groups. Thus, the at least one acid may be a carboxylic acid, such as one or more selected from the group comprising acetic acid, oxalic acid and tartaric acid. Dicarboxylic acids, such as oxalic acid are preferred.

In another embodiment, the at least one acid may comprise an acidified ammonia solution. The acidified ammonia solution may be prepared from the acidification of an aqueous ammonia solution with a strong acid. Preferably, the strong acid is one or more of the group comprising hydrochloric acid, hydrobromic acid, hydroiodic acid, nitric acid, sulphuric acid and p-toluene sulphonic acid, more preferably hydrochloric acid. The acidified aqueous ammonia solution can provide an aqueous ammonium salt. In a preferred embodiment, the ammonium salt of a strong acid may be selected from the group comprising ammonium nitrate, ammonium chloride and ammonium sulphate. The acid may be present in the aqueous solution at a concentration in the range of 0.01 to 3 M, preferably 0.05 to 2 M and more preferably 0.075 to 1.25 M.

The step of treating the spent molecular sieve catalyst with an aqueous solution stream may be carried out at a rejuvenation temperature of below 100° C., more preferably at a temperature in the range of from 70 to 90° C., still more preferably 80° C. The aqueous solution stream may be pre-heated to the rejuvenation temperature, or residual spent catalyst heat from the treatments in the reaction or regeneration zones may be relied upon.

The treating step may have a duration of a number of hours, typically from 1 to 2 hours for a batch-wise process. In a continuous process, the residence time of the spent molecular sieve catalyst in the rejuvenation zone may be up to 5 hours, more preferably in the range of from 1 to 2 hours, still more preferably 1 hour.

The treatment of the spent molecular sieve catalyst produces a spent aqueous solution, which can be removed from the rejuvenation zone as a spent aqueous solution stream. In a continuous process, the mass flow of the spent aqueous solution stream and aqueous solution comprising at least one acid can balance.

In order to ensure the separation of the spent aqueous solution from the rejuvenated molecular sieve catalyst, it may be washed with a wash solution comprising water in order to minimize the presence of any solid residue on the rejuvenated molecular sieve catalyst from the spent aqueous solution, particularly after heating. In order to minimize contamination of the rejuvenated molecular sieve catalyst by the wash solution, for instance by ion exchange between the molecular sieve in the catalyst and solvated species, de-ionized water is preferred.

The rejuvenated molecular sieve catalyst may then be heated to remove one or both of the spent aqueous solution and the wash solution. The heating step should raise the temperature of the rejuvenated molecular sieve catalyst to a level sufficient to evaporate the water from the spent aqueous solution and/or wash solution. In one embodiment, the heating may be carried out in the rejuvenation zone, for instance in a batch-wise rejuvenation process after the spent aqueous solution and wash solution have been drained. When the heating step is carried out in the rejuvenation zone, the temperature of the rejuvenation zone may be raised to above 100° C., more preferably in the range of from 105 to 200° C., still more preferably 120° C.

Alternatively, any further heating step may be carried out in the reaction or regeneration zones. This can be achieved by first removing rejuvenated molecular sieve catalyst from the rejuvenation zone, for instance as a fluidized stream such as a fluidized rejuvenated catalyst stream. The fluidized rejuvenated catalyst stream can be passed to the reaction or regenerations zones. In these zones, the heating step to remove one or both of the spent aqueous solution and wash solution may be carried out as part of the normal reaction process i.e. the OTO process or OCP in the reaction zone, or the regeneration process in the regeneration zone which have been discussed above.

### EXAMPLE

In these examples DME and 1-butene were reacted over oxygenate conversion catalysts comprising particles, the catalyst is formulated to contain 40 wt % of total zeolite (molecular sieve) and a matrix of 36 wt % kaolin clay (as filler) and 24 wt % of silica binder. Fresh catalyst was prepared by spray drying wherein the weight-based average particle size is between 70-90 μm. The catalyst was prepared by preparing an aqueous suspension containing MTT (ZSM-23 SAR 46) and the MFI (ZSM-5 SAR 280) zeolites with the clay and the binder, and subsequently spray drying the suspension. The MTT/MFI weight ratio is 80/20, based on total zeolite present in the catalyst.

Spent catalyst was obtained by exposing the catalyst to various hours at temperatures above 525° C. in a gaseous stream containing oxygenate (DME), 1-butene and steam. After exposing the catalyst to this feed, the catalyst was regenerated in an oxygen containing gas stream above 600° C. Afterwards, the catalyst was exposed to the feed again for several hours, after which the catalyst was regenerated again and the cycle was repeated.

In accordance to the process described herein, portions of the spent catalyst after regeneration were treated with different acidic solutions by suspending the catalyst in an aqueous solution containing the acid.

Typically, 15 grams of catalyst was used in 150 ml solution at 80° C. and stirred for 1 h, after which the suspension was allowed to cool to room temperature. After the treatment the samples were filtered, washed and subsequently dried at 120° C.

Prior to testing, a sample of the treated catalyst crushed and pressed into tablets and the tablets were broken into pieces and sieved. For catalytic testing, the sieve fraction of 60-80 mesh has been used.

Prior to test performance, all acid treated catalyst and the reference fresh catalyst were treated ex-situ in air at 500° C. for 2 hours.

In these examples methanol and 1-butene were used for conversion experiments at 525° C., at a Gas Hourly Space Velocity (GHSV) of 19000 ml.g_{zeolite}⁻¹.h⁻¹3 vol % 1-butene and 6 vol % methanol balanced in N₂. The catalyst was heated in N₂ to the reaction temperature and a mixture consisting of methanol, 1-butene balanced in N₂ was passed over the catalyst at atmospheric pressure (1 bar). Gas Hourly Space Velocity is based on total gas flow (ml. g_{zeolite}⁻¹.h⁻¹).

The effluent from the reactor was analyzed by gas chromatography (GC) to determine the product composition. The composition has been calculated on a weight basis of all hydrocarbons analyzed.

The catalyst activity shown in Table 1 was determined by examining the C5 concentration and comparing the concentration of C5 olefins in the product stream with that of the fresh catalyst at various gas hourly space velocities. The gas hourly space velocity for the spent or rejuvenated catalyst relative to the gas hourly space velocity of the fresh catalyst at equal C5 concentration gives the percentage of remaining activity of the spent or rejuvenated catalyst.

TABLE 1
Catalyst activity for fresh, spent and various rejuvenated catalysts

| | Catalyst activity compared |
|---|---|
| | to fresh catalyst (%) |
| Fresh catalyst (reference) | 100 |
| Spent catalyst (comparative) | 42 |
| After NH4/HCL rejuvenation | 70 |
| After acetic 0.1 M rejuvenation | 62 |
| After acetic 0.5 M rejuvenation | 60 |
| After oxalic 0.05 M rejuvenation | 75 |
| After oxalic 0.1 M rejuvenation | 88 |
| After oxalic 0.5 M rejuvenation | 92 |

Table 1 shows that acid treatment of a regenerated catalyst significantly increases catalyst activity compared to that of a spent catalyst after regeneration which has an activity of only 42% of that of a fresh catalyst. In addition, it is apparent that treatment with aqueous solutions of oxalic acid provides the greatest restoration of activity, returning the activity of the rejuvenated catalyst to within 10% of that of a fresh catalyst.

Table 2 shows the product distribution defined by the division of the mass of product by the sum of the masses of all products for three catalyst formulations prepared as discussed above: a fresh catalyst, a spent catalyst, and a rejuvenated catalyst which has been treated with 0.1 M oxalic acid.

TABLE 2
Product distribution for fresh catalyst, spent and catalyst and
catalyst rejuvenated by oxalic acid treatment.

| | Fresh | | Rejuvenated |
|---|---|---|---|
| | Catalyst | Spent catalyst | catalyst |
| | (Reference) | (Comparative) | (Invention) |
| Reaction conditions | | | |
| GHSV (ml/g_{zeolite}/h | 19000 | 19000 | 19000 |
| Temperature (° C.) | 525 | 525 | 525 |
| TOS (h) | 0.77 | 0.25 | 0.25 |
| Reactor effluent | | | |
| C2 = (wt %) | 10.05 | 6.81 | 9.74 |
| C3 = (wt %) | 43.98 | 41.95 | 44.58 |
| C4 = (wt %) | 29.73 | 30.74 | 30.11 |
| C5 = (wt %) | 8.23 | 15.86 | 9.36 |
| C6+* (wt %) | 6.37 | 3.27 | 3.75 |
| benzene (wt %) | 0.53 | 0.72 | 0.60 |
| total aromatics (wt %) | 1.24 | 0.80 | 1.77 |

| | |
|---|---|
| balance | methane, dimethyl ether, ethanol |

* excluding aromatics

It is apparent that the spent catalyst produces less desirable pentylenes at the expense of the C2-3 olefins. However, upon rejuvenation with a 0.1 M aqueous solution of oxalic acid, the activity of the rejuvenated catalyst for ethylene and propylene is restored, to within 5% of the level of ethylene manufacture for a fresh catalyst, with a corresponding reduction in the proportion of pentylene produced.

The person skilled in the art will understand that the present invention can be carried out in many various ways without departing from the scope of the appended claims.

What is claimed is:

1. A process for rejuvenating a spent molecular sieve catalyst, said process comprising at least the steps of:
(a) treating the spent molecular sieve catalyst with an aqueous solution comprising at least one acid to provide a rejuvenated molecular sieve catalyst and a spent aqueous solution;
(b) removing spent aqueous solution from the rejuvenated molecular sieve catalyst to provide the rejuvenated molecular sieve catalyst.

2. The process of claim 1 wherein the molecular sieve is selected from the group comprising silicoaluminophosphate and aluminosilicate.

3. The process of claim 2 wherein the molecular sieve is a 10-membered ring zeolite.

4. The process of claim 3 wherein the 10-membered ring zeolite comprises one or more of the group comprising a TON-type aluminosilicate, such as ZSM-22, a MTT-type aluminosilicate, such as ZSM-23, and MFI-type aluminosilicate, such as ZSM-5.

5. The process of claim 1 wherein the spent molecular sieve catalyst is at least part of a spent catalyst provided by one or both of the steps of:
(iii) reacting a feedstock comprising oxygenate in an oxygenate to olefin reaction zone in the presence of a molecular sieve catalyst to produce a spent molecular sieve catalyst and a reaction effluent comprising olefins; and
(iv) reacting an C4+ hydrocarbon feedstock comprising olefin in an olefin cracking process reaction zone in the presence of a molecular sieve catalyst to produce a spent molecular sieve catalyst and a reaction effluent one or both of ethylene and propylene.

6. The process of claim 5 wherein the spent molecular sieve catalyst has a catalytic activity of less than 80% of that of fresh molecular sieve catalyst, preferably less than 50%.

7. The process of claim 1 wherein the at least one acid comprises at least one carboxylic acid group, preferably at least two carboxylic acid groups.

8. The process of claim 7 wherein the at least one acid is selected from the group comprising acetic acid, oxalic acid and tartaric acid.

9. The process of claim 1 wherein the step of treating the spent molecular sieve catalyst comprises contacting the spent molecular sieve catalyst with the aqueous solution comprising at least one acid, the acid having a concentration in the range of from 0.01 to 3 M, preferably 0.05 to 2 M, more preferably 0.075 to 1.25 M.

10. The process of any of the preceding claims, wherein the step of removing spent aqueous solution from the rejuvenated molecular sieve catalyst comprises washing the rejuvenated molecular sieve catalyst with a solution comprising water.

11. The process of claim 1, wherein the spent molecular sieve catalyst comprises carbonaceous deposits, said process further comprising, before the step of treating the spent molecular sieve catalyst, the step of:
regenerating the spent molecular sieve catalyst to remove at least a part of any carbonaceous deposits by heating with a regeneration gas stream comprising oxidant.

12. A process for the preparation of an olefinic product, the process comprising the steps of:
(a) reacting a feedstock comprising oxygenate in an oxygenate to olefin reaction zone in the presence of a molecular sieve catalyst to produce a reaction effluent comprising olefins and a spent molecular sieve catalyst; and
(b) rejuvenating the spent molecular sieve catalyst in accordance with a process comprising:
i. treating the spent molecular sieve catalyst with an aqueous solution comprising at least one acid to provide a rejuvenated molecular sieve catalyst and a spent aqueous solution;
ii. removing spent aqueous solution from the rejuvenated molecular sieve catalyst to provide the rejuvenated molecular sieve catalyst.

13. A process for the preparation of olefinic product comprising one or both of ethylene and propylene, the process comprising the steps of:
(a) reacting a C4+ hydrocarbon feedstock comprising olefin in an olefin cracking process reaction zone in the presence of a molecular sieve catalyst to produce a spent molecular sieve catalyst and a reaction effluent comprising one or both of ethylene and propylene;
(b) rejuvenating the spent molecular sieve catalyst in accordance with a process comprising:
i. treating the spent molecular sieve catalyst with an aqueous solution comprising at least one acid to provide a rejuvenated molecular sieve catalyst and a spent aqueous solution;
ii. removing spent aqueous solution from the rejuvenated molecular sieve catalyst to provide the rejuvenated molecular sieve catalyst.
